(19) United States
(12) Patent Application Publication
(10) Pub. No.: US 2002/0077317 A1
(43) Pub. Date: Jun. 20, 2002

(54) Method of potentating the action of 2-methoxyoestradiol, statins and C-peptide of proinsulin

(76) Inventor: Undurti Narasimha Das, Norwood, MA (US)

Correspondence Address: Rama B Nath, 4000 Parkside Center Blvd #1805, Farmers Branch, TX, 75244, US

(21) Appl. No.: 09/737,671

(22) Filed: Dec. 15, 2000

(51) Int. Cl. A61K 38/28; A61K 39/395; A61K 31/56; A61K 31/40; A61K 31/35

(52) U.S. Cl. 514/171; 424/131.1; 514/4; 514/423; 514/460

(57) ABSTRACT

A method of stabilizing and potentiating the actions of 2-methoxyoestradiol, statins, H₂ blockers, and C-peptide of proinsulin which have modifying influence on angiogenisis and inhibiting the growth of tumor cells, peptic ulcer disease, diabete mellitus and its complications, and Alzheimer's disease as applicable by using in coupling conjugation certain polyunsaturated fatty acids (PUFAs) chosen from linoleic acid, gamma-linolenic acid, dihomo-gamma-linolenic acid, arachidonic acid, alpha-linolenic acid, eicosapentaenoic acid, docosahexaenoic acid, cis-parinaric acid or conjugated linoleic acid in predetermined quantities. Uncontrolled angiogenic activity and tumor growth can be inhibited by the selective use of a mixture of PUFAs with anti-angiogenic substances used selectively, and optionally in conjunction with predetermined anti-cancer drugs. A preferred method of administration of the mixture to treat a tumor is intra-arterial administration into an artery which provides the main blood supply for the tumor. The method will also be useful in the treatment of peptic ulcer disease, diabetes mellitus and its complications and Alzheimer's disease.

### RELATED APPLICATIONS

This invention relates to co-pending U.S. application Ser. No. 09/392,953 Filed on Sep. 9, 1999 and entitled “Method of Treatment for Cell Proliferative Disorders including Cancer”, and Ser. No. 09/478,291 Filed on Jan. 5, 2000 and entitled “A method of stabilizing and potentiating the action of anti-angiogenic substances”, which are incorporated herein by reference.

### FIELD OF THE INVENTION

The present invention generally relates to the use of 2-methoxyoestradiol (2-ME), an anti-angiogenic agent and a free radical inducer, in the cure of cell proliferative disorders including cancer and other disorders caused by uncontrolled angiogenic activity in the body. More particularly, the invention is directed to the efficacious use of 2-methoxyoestradiol and other anti-angiogenic, tubule binding, and enhancers of free radical generation agents.

### BACKGROUND OF THE INVENTION

The term angiogenesis refers to the generation or formation of new blood vessels into a tissue or organ. Angiogenesis can occur both during some physiological processes and/or in some pathological conditions. For example, angiogenesis can be seen to occur during wound healing, fetal growth, corpus luteum, and endometrium, etc., (1). Endothelial cells, which cause to form the inner lining of the blood vessels, are constituted by a thin layer of epithelial cells and these cells are necessary for the process of angiogenesis. During the process of angiogenesis, irrespective of whether it is physiological or pathological, the endothelial cells release enzymes which can produce erosions of the basement membrane through which the endothelial cells cause protrusions. In response to the stimuli given by various agents, endothelial cells proliferate and migrate through the protrusions and form a sprout of the parent blood vessel. These endothelial cell sprouts can merge to form capillary loops leading to the formation of new blood vessel(s). If the blood vessels are in a tumor area, these new blood vessels in turn will provide enough nutrients and energy sources so that tumor cells can divide, proliferate and grow both in number and size. Thus, the process of angiogenesis is both essential and critical to the growth of cancer. The other pathological states in which angiogenesis plays a critical role include: rheumatoid arthritis, psoriasis, scleroderma, myocardial angiogenesis, corneal diseases, diabetic retinopathy associated with neovascularization, macular degeneration, ovulation, menstruation etc. The process of angiogenesis also appears to be critical for tumor metastasis.

Since angiogenesis is such a critical process in the promotion of cancer and tumor metastasis, selective suppression of angiogenesis can lead to the inhibition of tumor growth. There are other situations where uncontrolled angiogenesis is undesirable. For instance, formation of new blood vessels in an area like cornea during the process of healing of the corneal ulcer, if it is in excess, can lead to corneal scar formation.

In the case of rheumatoid arthritis, angiogenesis can lead to continued inflammation in the joints and also to osteoporosis. In such an instance, prevention of formation of new blood vessels will lead to reduction in inflammation and also prevention of fibrous ankylosis and bony ankylosis. Thus, selective prevention and control of angiogenesis may be of benefit in the aforementioned conditions, as well as in several other conditions such as: uterine fibroids, psoriasis, scleroderma, diabetic retinopathy, keloids, ovulation etc. Another area where prevention of angiogenesis will be of benefit is in the inhibition of ovulation and menstruation and growth of placenta and this will lead to prevention of fertilization and growth of the fetal tissue respectively. This may, thus, form a new approach in the development of fertility control measures.

Angiostatin® and Endostatin®

Two naturally occurring molecules which have been identified to adversely influence or inhibit angiogenesis are angiostatin® and endostatin® (2). Both these molecules are proteins. Angiostatin® is a protein of molecular weight of approximately 38 kD and has an amino acid sequence substantially similar to that of a fragment of murine plasminogen beginning at amino acid number 98 of an intact murine plasminogen molecule. The amino acid sequence of angiostatin® varies only slightly between species and human angiostatin® is substantially similar to the murine plasminogen fragment. But, it may be mentioned here that the active human angiostatin® sequence starts either at the amino acid number 97 or 99 of an intact human plasminogen amino acid sequence. In addition, human plasminogen has potent anti-angiogenic activity even in a mouse tumor model. This explains why both murine and human plasminogens and angiostatin®/endostatin® molecules show fairly similar anti-angiogenic activities in a variety of animal tumor models (3).

U.S. Pat. No. 5,792,845 issued on Aug. 11, 1998 to O'Reilly et al teaches that therapies directed at control of the angiogenic process could lead to the abrogation or mitigation of certain diseases. O'Reilly et al suggests that modulation of the formation of capillaries in angiogenic processes (such as wound healing and reproduction) is useful since undesired and uncontrolled angiogenesis can cause certain diseases to progress. O'Reilly et al teaches that angiostatin® protein has the capability of inhibting angiogenesis, eg., to inhibit the growth of bovine capillary endothelial cells in culture in the presence of fibroblast growth factor.

U.S. Pat. No. 5,932,545 issued on Aug. 3, 1999 to Henkin et al teaches an anti-angiogenic drug in the form of a peptide or a salt thereof, to treat cancer, arthritis and retinopathy. The Henkin et al patent states however that angiogenesis inhibitors could cause systemic toxicity in humans.

Angiostatin® in the O'Reilly patent '845 is described and claimed as an isolated nucleotide molecule with a specific sequence. It has been stated however that the angiostatin® molecule as known at present is not suitable for clinical trials.

It is important to note that angiostatin® is derived from plasminogen or plasmin. It has been shown that human prostate carcinoma cell lines express enzymatic activity that can generate bioactive angiostatin® from purified human plasminogen or plasmin This bioactive angiostatin® has been shown to inhibit human endothelial cell proliferation, basic fibroblast growth factor-induced migration, endothelial cell tube formation, and basic fibroblast growth factor-induced corneal angiogenesis. In an extension of this study, it was noted that a serine proteinase is necessary for angiostatin® generation (4).

Angiostatin® derived from plasminogen, selectively inhibits endothelial cell proliferation. When angiostatin® is given systemically it shows potent inhibitory action on the growth of tumor and renders metastatic and primary tumors to go into a dormant state by striking a balance between the rate of proliferation and apoptosis of the tumor cells (5). The very identification of angiostatin® has come from the observation that when a primary tumor is present, the growth of metastases is suppressed. On the other hand, after tumor removal, the previously dormant metastases develop new blood vessels (neovascularization) and grow. Both serum and urine from the tumor-bearing animals, but not from controls, showed very specific inhibitory action on the growth of endothelial cells. Subsequent studies led to the purification of this inhibitor of endothelial cells which was later identified as a 38 kD plasminogen fragment namely angiostatin . It is now known that angiostatin®, which can also be obtained by a limited proteolytic digestion of human plasminogen, but not intact plasminogen can be administered systemically to block neovascularization and growth of metastases and primary tumors. A recombinant human angiostatin® which comprises of kringles 1-4 of human plasminogen (amino acids 93-470) expressed in Pichia pastoris has been prepared and is now available for use.

This recombinant angiostatin® showed the same physical properties as that of the natural angiostatin® in terms of molecular size, binding to lysine, reactivity with antibody to kringles 1-3 (3, 6). This recombinant angiostatin® when given to experimental animals, showed anti-angiogenic and anti-tumor activity (3). In adiition, recombinant mouse angiostatin® was produced using the baculo-virus infected insect cells (7), which also (the secreted protein) showed potent inhibitory action on the proliferation of bovine capillary endothelial cells in vitro. The conversion of plasminogen to angiostatin® by PC-3 cells is now identified to be due to two components released, urokinase (uPA) and free sulfhydryl donors (FSDs). This is supported by the fact that even in a cell-free system, angiostatin® can be generated from plasminogen by plasminogen activators (u-PA, tissue-type plasminogen activator, tPA or streptokinase) in combination with any one of free sulfhydryl donors such as N-acetyl-L-cysteine, D-penicillamine, captopril, L-cysteine, or reduced glutathione. This cell-free derived angiostatin® also showed anti-angiogen activity both in vitro and in vivo and suppressed the growth of Lewis lung carcinoma metastases (8).

Angiostatin® administration to mice with subcutaneous hemangioendothelioma and associated disseminated intravascular coagulopathy revealed that in addtion to a significant reduction in the size of the tumor, increased survival, decrease in thrombocytopenia and anemia was noted (9). This indicates that angiostatin® may also be useful to treat disseminated intravascular coagulopathy.

One of the mechanisms by which angiostatin® inhibits endothelial cell proliferation includes its ability to affect by 4 to 5 fold the expression of E-selectin in proliferating endothelial cells (10). On the other hand, angiostatin® did not alter cell cycle progression significantly. Further, angiostatin® also enhanced the adhesion activity in proliferating endothelial cells.

There is a close relationship between HME (human macrophage metalloelastase) and angiostatin® is understood since, metalloproteinase(s) can block angiogenesis® by converting plasminogen to angiostatin® (11,12,13).

Another mechanism by which recombinant human and murine angiostatins can block angiogenesis is by inducing apoptosis (programmed cell death) of endothelial cells (14), similar to that seen with tumor necrosis factor (TNF) and transforming factor-beta 1 (TGF-beta1), which are also known to induce apoptosis in endothelial cells.

Some of the factors which are known to inhibit the generation of angiostatin include TGF-beta1 and plasminogen activator inhibitor type-I (PAI-1), at least, by human pancreatic cancer cells in vitro (15).

Endostatin®, which is also similar to angiostatin®, has been shown to cause a dramatic reduction of primary and metastatic tumors in experimental animals. Endostatin® is a 20 kDa C-terminal fragment of collagen XVIII. Endostatin® could specifically inhibit endothelial cell proliferation and angiogenesis and thus, block tumor growth (2, 16).

Though both angiostatin® and endostatin® and other similar anti-angiogenic molecules provided an important therapeutic advance for cancer treatment, it should be emphasized here that the use of these chemicals for cancer are not without problems, especially angiostatin® used in the animal studies seem to be too high for clinical trials (17). Further, repeated injections and long-term treatment with angiostatin® are required to obtain its maximal anti-tumor effect. Further repeated injections of both angiostatin and endostatin can lead to the generation of antibodies against them, as they are antigenic in nature, which can effectively neutralize their actions and thus, decrease their anti-cancer efficacy (18). In view of this, methods to supplement the anti-angiogenic action of angiostatin® and endostatin® and other similar compounds are considered desirable. These methods include: use of angiostatin® along with other conventional anti-cancer drugs including radiation and novel methods of delivery of angiostatin® to tumor cells (19). Mauceri et al (20) studied the combined effect of radiation with angiostatin® and showed that this combination produced no increase in toxicity towards normal tissue. Both in vitro and in vivo studies showed that these agents (radiation and angiostatin®) in combination target the tumor vasuclature. In an extension of this study, Gorski et al (21) demonstrated that the efficacy of experimental radiation therapy is potentiated by brief concomitant exposure of the tumor vasculature to angiostatin®.

Two novel methods of delivery of angiostatin® and similar compounds to the tumor cells that have been tried include:

(a) Nguyen et al (22) generated recombinant adeno-asscoaited virus (rAAV) vectors that carry genes encoding for angiostatin®, endostatin®, and an antisense mRNA species against vascular endothelial growth factor (VEGF). These rAAVs efficiently transduced three human tumor cell lines that have been tested. Further, testing of the conditioned media from cells transduced with this rAAV or with rAAV-expressing endostatin® or angiostatin® inhibited effectively endothelial cell proliferation in vitro. These results indicate that rAAVs can be used to block angiogenesis and cancer growth.

(b) In a different approach, Chen et al (23) examined whether liposomes complexed to plasmids encoding angiostatin® or endostatin® can inhibit angiogenesis and growth of tumors. These studies revealed that plasmids expressing angiostatin® (PCI-angio) or endostatin® (PCI-endo) can effectively reduce angiogenesis and the size of the tumors implanted in the mammary fat pad of male mice to a significant degree. In addition, liposomes complexed to PCI-endo when given intravenously reduced tumor growth in nude mice by nearly 40% when compared to controls (23).

2-methoxyoestradiol (2-ME)

Another chemical that is present in the body that is a natural metabolic byproduct of the hormone, estradiol, by its hydroxylation followed by O-methylation in the liver, is 2-methoxyoestradiol (2-ME). In 1992, Lottering et al showed that 2-ME has cytotoxic action on breast cancer cells, MCF-7 (24). Studies have suggested that 2-ME can interfere with spindle formation, inhibit DNA synthesis and suppres the growth of Chinese Hamster V79 cells in culture (25). It has also been shown that 2ME can inhibit endothelial cell migration and angiogenesis both in vitro and in vivo (26). 2-ME did not have any effects on normal human skin fibroblasts (26). It was observed that even at 100 micro M concentration 2-ME had no effect on normal human skin fibroblasts, where as the half-maximal inhibitory concentration of the compound (IC₅₀) is 0.15 micro M for endothelial cells (26). Other studies showed that oral administration of 2-ME in mice can inhibit capillary formation in solid tumors and reduce their growth. In vivo studies have also indicated that the anti-tumor activity of 2-ME is not associated with general toxicity (26). In another set of investigations, it was observed that 2-ME can induce the accumulation of p53 protein, which is known to facilitate apoptosis (programmed cell death) (27). But, studies performed by Huang et al (28) showed that 2-ME can induce the death of tumor cells which do not express p53 also. This suggests that both p53 expressing and p53 non-expressing tumor cells can be killed by 2-ME. Further studies showed that 2-ME is able to bind to SOD (superoxide dismutase) and thus, enhance free radical generation in the tumor cells, which in turn is responsible for its cytotoxic action on tumor cells. These results are interesting since, earlier the investigator showed that polyunsaturated fatty acids (PUFAs) can kill the tumor cells by enhancing free radical generation in the tumor cells but not in the normal cells (29). PUFAs also can suppress the activity of SOD in the tumor cells (30), but the exact mechanism is not clear, though it is possible that PUFAs might also bind to SOD and thus inactivate and/or inhibit its (SOD) action. PUFAs seem to enhance free radical generation in the cells by activating NADPH oxidase, an enzyme that is necessary for generating free radicals (31). It is evident from this discussion that 2-ME enhances free radical generation by binding to and inhibiting the activity of SOD, which normally quenches superoxide anion in the cells, where as PUFAs enhance free radical generation in the tumor cells both by suppressing the activity of SOD and also by activating NADPH oxidase. Further, PUFAs can also enhance the expression of p53 (32) similar to 2-ME. It is known that p53 is also capable of enhancing free radical generation in the cells i.e. it has pro-oxidant actions (33). Thus, there is a close association between p53, 2-ME and various PUFAs in bringing about their killing effect on cancer cells.

C-Peptide of Proinsulin

It is also important to mention here that another peptide called as C-peptide of proinsulin also seems to have actions similar to 2-ME and polyunsaturated fatty acids. For instance, C-peptide of proinsulin can bind to endothelial cells, stimulate Na⁺-K⁺-ATPase activity, enhance nitric oxide generation from the endothelial cells and is believed to be useful in the prevention of long-term complications of diabetes mellitus such as neuropathy, angiopathy, retinopathy, nephropathy etc. Studies performed by the inventor showed that PUFAs (polyunsaturated fatty acids) can enhance Na⁺-K⁺-ATPase activity, enhance nitric oxide synthesis and are also probably useful in the prevention and treatment of long-term complications of diabetes mellitus (34, 35). Several recent studies have suggested that C-peptide may be useful in the prevention of long term complications due to diabetes mellitus (36, 37).

Statins

It is also pertinent to mention here that drugs called as statins, which inhibit the activity of the enzyme HMG-CoA-reductase (3-hydroxy-3-methylglutaryl-CoA reductase), also have actions similar to C-peptide and PUFAs. HMG-CoA reductase is the rate limiting step in cholesterol biosynthesis and hence, are used to lower cholesterol and prevent the development of atherosclerosis. Recently, it has been recognized that even statins can also enhance nitric oxide synthesis, inhibit tumor cell growth and induce apoptosis of tumor cells in vitro (38), actions which are similar to those of C-peptide and PUFAs since these chemicals also enhance nitric oxide synthesis. Further, both statins and PUFAs can inhibit the production of pro-inflammatory cytokines such as tumor necrosis factor α (TNFα) (39, 40). Further, TNFα is toxic to neurons and statins by inhibiting the production of TNFα may promote their survival and prevent Alzheimer's disease and improve memory. It was also observed that statins enhance the expression of BMPs (bone morphogenetic proteins) such as BMP-2, BMP-4, BMP-6, BMP-7, BMP-9, and BMP-12 which induce and maintain the neuronal cholinergic phenotype in the central nervous system and thus, statins help maintain memory.

### SUMMARY OF THE INVENTION

All the above factors and observations attest to the fact that malignant tumors are angiogenesis-dependent diseases and that tumor cells can be killed both by using anti-angiogenic substances, by enhancing free radical generation in them or by both mechanisms. But, it should be mentioned here that tumor-associated angiogenesis is a complex, multi-step process which can be controlled by both positive and negative factors. It appears, as though, angiogenesis is necessary, but not sufficient, as the single event for tumor growth (41). But, it is evident from several experimental results that angiogenesis may be a common pathway for tumor growth and progression. Though several anti-angiogenic agents are being tried to arrest tumor growth, these are not without problems. Some of these agents such as angiostatin® and endostatin® are proteins/peptides and their long-term use may lead to the development of antibodies which can neutralize their action. Further, both angiostatin® and endostatin® are unstable and are difficult to produce in large amounts. These have to be given for more than 6 months to 1 year to obtain substantial regression of the tumor and that too parenterally. Even then, the response of various cancers to these drugs is doubtful. Some other compounds like 2-ME are metabolites of hormones and the long-term consequences of their use is not known.

In view of this, it is desirable and necessary to make efforts to stabilize and potentiate the actions of known anti-angiogenic molecules and of chemicals that may have both anti-angiogenic and direct tumoricidal action.

The present invention teaches the efficacious use of anti-angiogenic substances including 2-ME, which can inhibit endothelial cell proliferation by coupling them to cis-unsaturated fatty acids, which also have anti-angiogenic and cytotoxic actions on tumor cells, such that the actions of these substances are potentiated. Further, as angiogenesis is involved in other disease processes such as inflammation, tumor metastasis, etc., it is envisaged that the conjugate(s) of anti-angiogenic substances and PUFAs will be useful in these diseases also.

Since, both C-peptide and PUFAs have similar actions and since, in diabetes the plasma and tissue levels of various PUFAs are decreased, it is suggested that a combination of PUFAs and C-peptide or its analogues or its carboxy-terminal tetra and pentapeptides may be used to prevent the complications of diabetes mellitus.

There are many similarities in the actions of statins, PUFAs and C-peptide of proinsulin. This indicates that these 3 molecules: c-peptide of proinsulin, PUFAs and statins, interact with each other very closely or one may regulate the action(s) of the other molecule. Thus, it is suggested that when these chemicals or agents are given together their combined effect will be more than the action of each molecule when used alone. It is also emphasized here that PUFAs are admixed or conjugated to anti-angiogenic proteins/peptides, 2-ME and C-peptide of proinsulin not only as a carrier of these later chemicals but also to potentiate their action. It is also pertinent to mention here that when these proteins/peptides or 2-ME when admixed or conjugated to PUFAs and only then a substantial and highly desirable anticancer, anti-angiogenic and anti-diabetic actions of these compounds is seen.

In this context, it is important to note that the inventor found that polyunsaturated fatty acids (PUFAs) such as gamma-linolenic acid (GLA), dihomo-GLA (DGLA), arachidonic acid (AA), eicosapentaenoic acid (EPA) and docosahexaenoic acid (DHA) can selectively kill the tumor cells (31, 32, 45-50) and under specific conditions and in conjugation with salts such as lithium and a lymphographic agent these fatty acids can actually behave as anti-angiogenic substances, i.e. they block all the blood supply to the tumor but not to the normal tissues and also prevent generation of new blood vessels. This interruption of blood supply to the tumor is not a physical obstruction as is found with embolization but is a chemical block (in that the blood vessel is stimulated to contract such that the blood supply to the tumor is blocked). Using these fatty acids in this particular combination, the inventor has successfully treated human hepatocellular carcinoma and giant cell tumor of bone with few or no side-effects.

Described hereinafter is a novel combination of a protein/peptide(s) or a chemical and a lipid and method(s) for its use. The protein(s) or the chemical referred to herein is a potent and specific inhibitor of endothelial proliferation and angiogenesis and an inhibitor of tumor cell growth. The lipid may be one or more of the polyunsaturated fatty acids: LA (linoleic acid), GLA, DGLA, AA, ALA (alpha-linolenic acid), EPA, DHA and cis-parinaric acid. In this instance or method the polyunsaturated fatty acid combination needs to be given to a patient only once or at the most twice within a period of 1 to 2 months.

This invention teaches that unlike angiostatin®/endostatin®, and 2-ME, these fatty acids are not only cytotoxic to the tumor cells but are also able to function as anti-angiogenic agents. Further, polyunsaturated fatty acids when given in the formulated form along with 2-ME, statins, C peptide of proinsulin will be more potent in view of their anti-angiogenic and anticancer actions. The inventor has discovered that when PUFAs are given in combination with C-peptide proinsulin or statins (either as a mixture or in conjugated form or as a complex), and only then such a combination would not only prevent the growth of the tumor cells or even kill the tumor cells but also that such a therapy would be useful in the prevention and treatment of diabetes mellitus and even in the prevention of long-term complications of diabetes mellitus.

The invention in one aspect teaches a method of interrupting blood supply to a tumor region, but not to the normal tissues, causing necrosis or apoptosis and also killing the tumor cells more specifically. The invention also provides a method of causing anti-angiogenic action in the tumor region with the result that new blood vessels and collaterals are not formed to sustain the tumor. The present invention in another aspect tackles the issue of drug delivery to the target tissue and provides the most efficacious method of administering an admixture of selected PUFAs with other elements such as 2-ME, statins, C-peptide of proinsulin, which have anti-angiogenic and tumoricidal actions as will be described in more detail hereinafter.

The invention in yet another aspect teaches a method of interrupting blood supply to a tumor using a pre-determined admixture of at least a PUFA and an anti-angiogenic agent such as 2-ME causing necrosis with very desirable results. Both the PUFAs and 2-ME, statins or C-peptide of proinsulin compounds being similar in function, the invention also provides a method of causing anti-angiogenic action in the tumor region with the result that new blood vessels and collaterals are not formed to sustain the tumor in the tumor region treated according to the invention. The present invention in another aspect tackles the issue of drug delivery to the target tissue and provides the most efficacious method of administering an admixture of selected PUFAs along with selected ones of 2-ME, statins or C peptide of proinsulin and other elements as will be described hereinafter.

The invention in another aspect also teaches a method of using an admixture of at least a PUFA and 2-ME, C-peptide proinsulin and/or statins to prevent or treat diabetes mellitus and its long-term complications. Since, PUFAs and 2-ME, C-peptide proinsulin and/or statins have similar function, the invention also provides a method of preventing or treating osteoporosis, Alzheimer's disease and preventing and/or treating long-term complications of diabetes such as nephropathy, vasculopathy, neuropathy, etc. Such a combination of PUFAs and 2-ME, C-peptide proinsulin and/or statins alone will kill tumor cells selectively and /or show anti-angiogenic action as well. Even cimetidine, a H₂ blocker, which is used in the treatment of peptic ulcer, is also known to have anti-agiogenic action (48). The inventor found that other H₂ blockers such as famotidine etc., will also have similar anti-angiogenic action. Since, PUFAs also have anti-angiogenic action, it is noted that a combination of PUFAs+H₂ blockers (given as a mixture, conjugate, or as a complex) will potentiate each others action and hence, such a combination will ultimately have a potent anti-angiogenic action. In view of this, such a mixture of PUFA(s)+H₂ blocker(s) will serve as a potent anti-cancer drug by virtue of their (or its) anti-angiogenic action and other similar actions. In addition, the inventor has also shown earlier that PUFAs have ability to reduce stomach acid production and heal peptic ulcers both in experimental animals and humans (49-51). Hence, it is suggested that such a combination of H₂ blocker+PUFAs will also possess potent ulcer healing property and so will be useful in the treatment of peptic ulcer disease as well.

The invention in another aspect also teaches a method of using an admixture of PUFAs (especially a combination of arachidonic acid, eicosapentaenoic acid and docosahexaenoic acid) and at least one of statins to prevent or treat Alzheimer's disease and to improve memory. Both PUFAs and statins inhibit the production of pro-inflammatory cytokines such as tumor necrosis factor α (TNFα) and interleukin-6 (IL-6) (39, 40) and thus, they show anti-inflammatory actions. It is known that heightened expression of TNFα can decrease the capacity of insulin-like growth factor1 (IGF-1) to promote survival of neurons (52). Hence, by suppressing the production of TNFα, both PUFAs and statins promote the survival of neurons and thus, not only prevent Alzheimer's disease but also improve memory.

Both statins and PUFAs are potent inhibitors of HMG-CoA reductase, the rate limiting enzyme of the mevalonate pathway. Mevalonate is the precursor of cholesterol and a variety of isoprenoid containing compounds. It is known that isoprenoid precursors are necessary for the post-translational lipid modification (called as prenylation) and hence, the function of Ras and other small GTPases. Small GTPases have a negative control on the expression of BMPs, especially BMP-2. Thus, inhibition of the mevalonate pathway by statins and PUFAs prevent the function of small GTPases and enhance the expression of BMP-2 and other BMPs. BMP-2, BMP-4, BMP6, BMP-7, BMP-9 and BMP-12 are known to induce and maintain the neuronal cholinergic phenotype in the central nervous sytem (53). Acetylcholine is known to participate in memory consolidation. Further acetylcholine is a potent stimulator of nitric oxide synthesis. Hence, a rational combination of statins and PUFAs can prevent dementia, Alzheimer's disease and improve memory by increasing nitric oxide synthesis, inhibiting the production of TNFα and enhancing the concentrations of various BMPs in the brain.

Further, another modality of therapy that is currently under evaluation to kill tumor cells is the use of specific monoclonal antibodies that target the specific tumor cell antigens. These monoclonal antibodies are given by themselves or in conjugation with toxins such as cholera toxin etc., or conventional anti-cancer drugs. But, this modality of therapy has not been very successful. Since these monoclonal antibodies are targeted to the tumor cell antigens, the inventor has found that when these antibodies are coupled or complexed or given along with PUFAs, their ability to kill the tumor cells will be very effective.

Tumor cells are deficient in phospholipase A2, an enzyme necessary for the release of various PUFAs from the cell membrane lipids as a result of which the production of anti-neoplastic PGs such as PGD2 are not elaborated. In addition, tumor cells secrete an excess of PGE2, an immunosuppressive and mutagenic substance. Further, tumor cells are deficient in PUFAs such as GLA, AA, EPA and DHA due to the low activity of delta-6-desaturase. As a result of these metabolic changes, tumor cells are able to effectively circumvent body's defense and survive. The present invention provides a method of causing necrosis of tumor cells despite their known survival pattern.

Anti-Cancer Actions of PUFAs:

Tumor cells are not only deficient in PUFAs but also have low rate(s) of lipid peroxidation and low amounts of superoxide dismutase (SOD), and contain relatively large amounts of antioxidants such as vitamin E. It is also believed that low rates of lipid peroxidation and consequent low amounts of lipid peroxides in the cells can contribute to an increase in the mitotic process which ultimately leads to an increase in cell proliferation. Thus, a deficiency of PUFAs, high amounts of antioxidants and the presence of low amounts of lipid peroxides in the tumor cells can contribute to the growth of tumor cells. This is supported by studies by the inventor wherein it was noted that PUFAs such as GLA, DGLA, AA, EPA and DHA can decrease tumor cell proliferation. In addition, it was also observed that when appropriate amounts of GLA, DGLA, AA, EPA and DHA were administered to tumor cells and normal cells, obtained from American Type Culture Collection, only tumor cells were killed without having any significant action on the survival of normal cells in vitro. In mixed culture experiments, in which both normal and tumor cells were grown together, GLA showed more selective tumoricidal action compared to AA, EPA and DHA though, these latter fatty acids were also effective to some extent. This indicated that selective delivery of GLA, DGLA, AA, EPA and DHA to tumor cells may offer a new therapeutic approach in the treatment of cancer.

These in vitro results are supported by in vivo studies performed in animal tumor models. For example, it was noted that GLA, DGLA, AA, EPA and DHA when used either in the form of pure fatty acid alone or in the form of fatty acid rich oils could inhibit the growth of skin papilloma in mice, formation and growth of hepatoma in rats and ascitic tumor cells in the peritoneum of experimental animals. These results indicate that these fatty acids can inhibit the growth of a variety of tumors even in vivo. In further studies, it was noted that these fatty acids are able to enhance free radical generation and the lipid peroxidation process selectively in the tumor cells (similar to 2-ME) but not so much in the normal cells and thus, are able to bring about their cancer killing action.

This ability of PUFAs to augment free radical generation and lipid peroxidation in the tumor cells is analogous to the anti-tumor action of lymphokines such as tumor necrosis factor (TNF) and interferon (IFN), both alpha and gamma varieties and 2-ME. These lymphokines (also referred to as cytokines) are capable of inducing the release of PUFAs from the cell membrane lipid pool and enhance free radical generation in the cells. Similarly several anti-cancer drugs such as, but not limited to, doxorubicin and vincristine have the capacity to augment free radical generation and promote lipid peroxidation. In addition, PUFAs and their products can modulate immune response, augment a respiratory burst of neutrophils and free radical generation by macrophages. This evidence is further testified by the observation that the incidence of cancer in Eskimos is low as influenced by their traditional diet, which is rich in EPA and DHA. Inventor's studies have shown that PUFAs can be exploited as possible anti-cancer agents either alone or in combination with lymphokines and traditional anti-cancer drugs.

In a series of investigations by the inventor, it was also observed that the cytotoxic action of anti-cancer drugs such as doxorubicin, vincristine and cis-platinum can be augmented by various PUFAs such as GLA, DGLA, AA, EPA and DHA. In addition, these fatty acids could also enhance the cellular uptake of these anti-cancer drugs by the tumor cells and thus, are able to potentiate the anti-cancer actions of these drugs. In another similar experiment by the inventor, it was also observed that GLA, DGLA, AA, EPA and DHA were able to kill TNF resistant L-929 tumor cells in vitro. Further, these TNF-resistant tumor cells were rendered TNF sensitive by prior treatment of these L-929 cells by GLA, DGLA, AA, EPA and DHA.

These results indicate that PUFAs can not only kill the tumor cells by themselves but are also capable of potentiating the cell killing effect of various anti-cancer drugs, lymphokines such as TNF and IFN and also render anti-cancer drug and TNF-resistant tumor cells sensitive to the cytotoxic action of various anti-cancer drugs and lymphokines. In a similar fashion, it is noted that a combination of various PUFAs and 2-ME, statins, H₂ blockers and/or C-peptide proinsulin will all be able to kill the tumor cells either by a direct action and/or by their anti-angiogenic action.

In another set of experiments, it was also noted that vincristine resistant tumor cells, KB-Ch^{R}-8-5 can be made sensitive to the cytotoxic action of vincristine by GLA, DGLA, AA, EPA and DHA. Further, when sub-optimal doses of vincristine and fatty acids were added together to these vincristine resistant cells produced optimal (i.e. significant) cell killing action. This shows that vincristine and other anti-cancer compounds and PUFAs when added together to cancer cells, will potentiate the cytotoxic action of each other. Fatty acid analysis of both vincristine sensitive (KB-3-1) and resistant KB-Ch^{R}-8-5 cells revealed that the resistant cells have low amounts of GLA, AA, EPA and DHA compared to the vincristine sensitive tumor cells indicating that a deficiency of these fatty acids may be responsible for their resistance to the cytotoxic actions of anti-cancer drugs. Since, both vincristine sensitive and resistant tumor cells are easily (and to the same extent) killed by various PUFAs in vitro, this demonstrates that even drug-resistant tumor cells can be killed by these fatty acids.

In yet another set of experiments, the inventor also noted that L-929 cells which are resistant to the cytotoxic action of tumor necrosis factor (referred to as TNF-resistant L-929 cells) can also be made sensitive to the cytotoxic action of TNF by pre-treating these cells with various PUFAs. In other words, L-929 cells which are resistant to the cytotoxic action of TNF can be sensitized to the cytotoxic action of TNF by PUFAs. This again indicates that PUFAs can not only kill the tumor cells but can also serve as sensitizing agents rendering various tumor cells responsive to the cytotoxic action of various anti-cancer drugs and lymphokines (cytokines) such as tumor necrosis factor. These results suggest that when anti-cancer chemicals such as 2-ME, statins etc., are used together with PUFAs such a combination will be able to kill even drug resistant tumor cells.

It is to be noted in this context that PUFAs can bind to albumin and other proteins and hence, if given intravenously may not be available to be taken up by the tumor cells and consequently may not be able to bring about their cell killing action on the tumor cells. Though it is possible that when a combination of PUFA+2-ME, statins or C-peptide of proinsulin are given this binding property of PUFAs to albumin or other proteins may not be seen or is much less reduced. Neverthless, it is desirable that PUFAs should be delivered to the patients in such a manner that they are easily available to the tumor cells and are delivered selectively to the tumor cells. It is highly desirable that PUFAs including GLA+2-ME, statins, H₂ blocker or C-peptide of proinsulin be given intra-tumorally as was experimentally done in the case of human gliomas, or, intra-arterially by selective intra-arterial infusion as was done experimentally in the case of hepatoma and giant cell tumor of the bone. But, it is also possible that in some cases of cancer such as Hodgkin's and non-Hodgkin's lymphoma wherein the tumor cells are extremely sensitive to the cytotoxic actions of PUFAs, even oral administration may be sufficient as was observed by the inventor in certain patients. Since, PUFAs can potentiate the cell killing effect of anti-cancer drugs and lymphokines, it is desirable to administer a combination of PUFAs, anti-cancer drugs, lymphokines such as TNF and interferon or other anti-angiogenic agents or chemicals such as 2-ME, statins, H₂ blocker or C-peptide of proinsulin or a combination thereof with or without a carrier agent such as an oily lymphographic agent as the situation indicates. Further studies have also revealed that PUFAs such as GLA, DGLA and EPA can prevent or ameliorate the side effects of anti-cancer agents such as gamma-radiation and cis-platinum to the bone marrow cells of mice. Thus, it appears that when PUFAs and conventional anti-cancer drugs/agents are given together, they not only potentiate the cytotoxic action of each on the tumor cells and thus, produce a synergistic and/or additive action in their ability to eliminate the tumor cells but it will also lead to elimination, reduction or amelioration of the side effects of conventional anti-cancer agents. Since PUFAs are able to potentiate the cytotoxic acton(s) of conventional anti-cancer agents and lmphokines, it is also seen that this will lead to a significant reduction in the doses of these latter agents without compromising the ultimate benefit namely, selective elimination of tumor cells or the tumor. In a similar way, a combination of 2-ME, statins, H₂ blockers and/or C-peptide of proinsulin+PUFAs will also show potent anti-cancer action both by direct action on the tumor cells and also by their anti-angiogenic action.

Some of the phenomena which reduce the efficacy of the cytotoxic action of PUFAs and conventional anti-cancer drugs/agents when used singly in vivo as compared to in vitro results include the following:

a. PUFAs or other chemicals when administered orally or intravenously can bind to albumin and other proteins in living beings and may not be available to be taken up by the tumor cells. But this ability of PUFAs to bind to proteins is made use of in the present invention and is detailed below.

b. The cytotoxic action of PUFAs or 2-ME is produced by the augmentation of free radical generation and lipid peroxidation in only tumor cells (but not in normal cells). The intensity of the cytotoxic action is disadvantageously reduced in actual clinical efforts because of inefficient transportation of the fatty acids or other anti-cancer molecules to the target areas.

c. Continued blood supply to tissue with proliferative cell disorders is not conducive to bringing about a significant amount of necrosis especially if the malignant cells multiply faster than they are being destroyed.

d. It was found from a study reported in a June, 1994 “Cancer letters” publication authored by N. Madhavi and U. N. Das that antioxidants like vitamin E and the superoxide anion quencher, superoxide dismutase (SOD) could completely inhibit free radical generation and lipid peroxidation generated by PUFAs like GLA, EPA and DHA. It appears that selective drug delivery to the target tissue will be conducive to the efficacy of the beneficial action of the PUFAs.

The present invention in one aspect resides in a method of inhibiting blood supply to a tumor only but not to normal tissues by using two types of substances: one a lipid and the other a protein or a peptide or 2-ME or a statin or a H₂ blocker or more than one of these compounds which have potent tumoricidal action and/or anti-angiogenic action. In addition, the invention also comprises of the steps of: locating an artery which carries major blood supply to the tumor, said artery being one that is proximate to the tumor, and intra-arterially injecting into the located artery a predetermined quantity of a polyunsaturated fatty acid (PUFA) in the form of a solution of at least one PUFA chosen from LA, GLA, DGLA, AA, ALA, EPA, DHA and cis-parinaric acid in combination with a protein/peptide, 2-ME, statin or H₂ blocker.

The invention in another aspect resides in a method for treating tumors and for facilitating visualization of remission of the tumor in response to treatment, comprising the steps of

(a) locating an artery which carries a major portion of blood supply to the tumor and is adjacent to the tumor;

(b) obtaining an initial radiographic image of the tumor region;

(c) injecting into the artery a mixture of (i) an oily lymphographic agent,

(ii) a lithium salt solution of at least one PUFA chosen from LA, GLA, DGLA, AA, ALA, EPA, DHA; and cis-parinaric acid

(iii) an anti-angiogenic protein/substance, 2-ME, a statin or a H₂ blocker which is co-valently linked to the fatty acid or from a mixture (fatty acid+anti-angiogenic protein or peptide, 2-ME, H₂ blocker or C-peptide of proinsulin).

(d) obtaining second and subsequent radiographic images of the tumor regions after predetermined lapses of time; and comparing the initial radiographic images with the second and subsequent radiographic images to assess the extent of remission of the tumor.

The invention in another aspect resides in a method of causing necrosis in a cancerous tumor by inhibiting blood supply only to the tumor but not to the normal tissues, and also by direct cytotoxicity to the tumor cells, comprising the steps of:

(a) locating an artery proximate to the tumor which carries major blood supply to the tumor;

(b) injecting into the located artery a mixture of (i) an anti-angiogenic protein/peptide, 2-ME, a statin, C-peptide of proinsulin or a H₂ blocker; (ii) a lithium salt solution of at least one essential fatty acid chosen from LA,GLA, DGLA, AA, ALA, EPA, DHA and cis-parinaric acid or conjugated linoleic acid (CLA).

(c) waiting for a predetermined time period and assessing a degree of necrosis in the tumor by examining by a radiographic study or by other means; and

(d) repeating step (b) if necessary to increase the necrosis.

In yet another aspect, the invention resides in a method of treating a glioma and visualizing remission of the glioma as it responds to treatment, comprising:

(a) obtaining an initial radiographic image of a region containing the glioma;

(b) injecting into the glioma region an admixture of (i) a sodium salt or any other suitable salt solution of at least one polyunsaturated fatty acid chosen from LA, GLA, DGLA, AA, ALA, EPA, DHA and cis-parinaric acid or a combination there of along with C-peptide of proinsulin, 2-ME, a statin or H₂ blocker;

(c) obtaining second and subsequent radiographic images of the glioma region after predetermined lapses of time; and comparing the initial radiographic pictures which shows the glioma, with second and subsequent radiographic images of the glioma region to visualize and assess the extent of remission of the glioma.

In yet another aspect, the invention resides in a method of treating mammalian cell proliferative disorders using an emulsion of a lithium salt of a PUFA or combinations of PUFAs and a predetermined anti-angiogenic protein/peptide, 2-ME, a statin, a H₂ blocker or C-peptide of proinsulin administered parenterally including a subcutaneous route. Preferably, the intra-arterial administration of the admixture containing PUFA(s) is done through a catheter. Also, the artery carrying major blood supply to the tumor is to be understood herein as synonymous to the artery which will supply the tumor feeding vessels.

Owing to a phenomenon which is consequent to inhibiting blood supply, the present invention makes it not conducive to the formation of new blood vessels i.e. angiogenesis. The anti-angiogenic substance or the anti-cancer substance in different implementations of this invention may be endostatin® or angiostatin® or 2-ME, a statian, H₂ blocker or C-peptide of proinsulin or a combination thereof.

### BRIEF DESCRIPTION OF THE ILLUSTRATIONS

A more detailed understanding of the invention may be had from the following description of preferred embodiments, given by way of example, and to be understood in conjunction with the accompanying illustrations/drawings wherein:

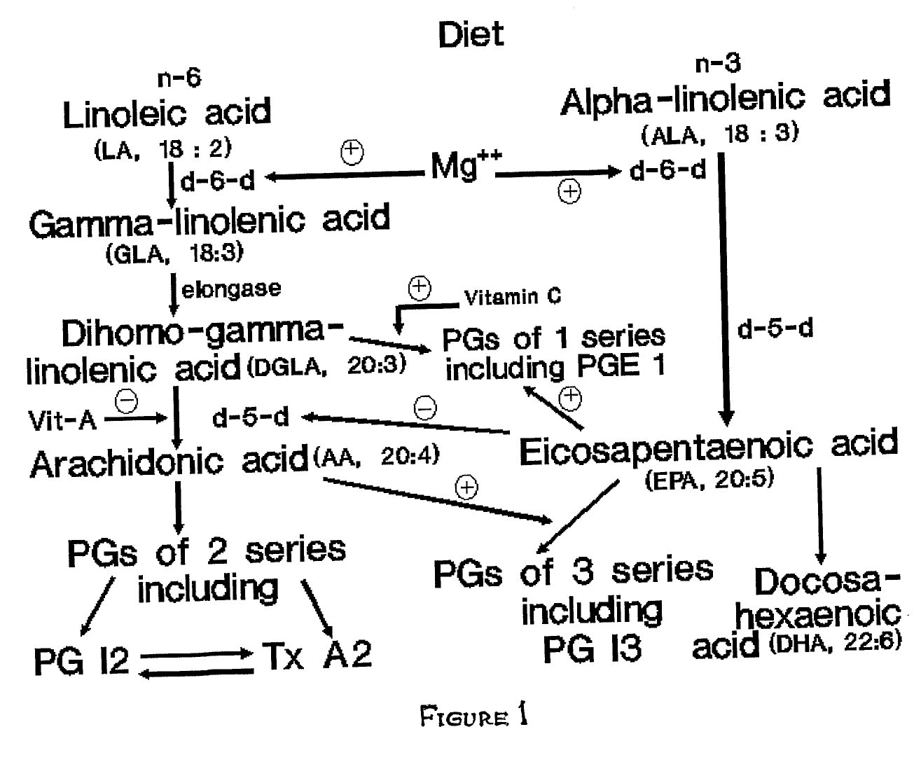

FIG. 1 illustrates the structural metabolism of essential fatty acids.

### DETAILED DESCRIPTION

FIG. 1 shows a typical known metabolism pattern of essential fatty acids as known in prior art. Essential fatty acids are precursors of eicosanoids and are important structural components of cell membranes. They also provide the substrates for the generation of lipid peroxidation products which have an inhibitory action on cell proliferation. Tumor cells are known to have low delta-6-desaturase activity, an enzyme necessary for the desaturation of dietary linoleic acid (LA, 18:2, n-6) and alpha-linolenic acid (ALA, 18:3, n-3) to their respective products. In an earlier study, the inventor has shown that hepatocarcinogens, diethylnitrosamine (DEN) and 2-acetylaminofluorine (2-AAF), can suppress the activity of delta-6-desaturase and delta-5-desaturase resulting in low levels of gamma-linolenic acid (GLA, 18:3, n-6) and arachidonic acid (AA, 20:4, n-6) and eicosapentaenoic acid (EPA, 20:5, n-3) and docosahexaenoic acid (DHA, 22:6, n-3) in the tumor cells. These results led the inventor and others to study the effect of various fatty acids on the survival of tumor cells in vitro. Addition of EFAs (LA and ALA) and other PUFAs such as GLA, DGLA, AA, EPA, DHA and cis-parinaric acid to a variety of tumor cells in vitro showed that only tumor cells are killed by these fatty acids without harming the normal cells. This selective tumoricidal action of fatty acids seems to be mediated by free radicals and lipid peroxides.

Similar to these fatty acids, radiation, some anti-cancer drugs and cytokines (lymphokines) also seem to have the ability to generate free radicals in tumor cells and thus, bring about their tumoricidal actions.

Since drug resistance is a major obstacle in the clinical treatment of cancer and as PUFAs have selective tumoricidal action, the inventor studied the effects of PUFAs on drug-resistant tumor cells and their modulating influence on the actions of anti-cancer drugs.

In the above context, in addition to producing reversal of tumor cell drug resistance by the administration of polyunsaturated fatty acids, it is seen from the invention that the manner of targeting the cancerous tissue is very critical to the efficacy and the speed with which necrosis can be brought about. More particularly, it is realized through this invention that by delivering a chosen admixture of salts of predetermined polyunsaturated fatty acids and predetermined anti-angiogenic substance(s) or 2-ME or statins or a H₂ blocker or C-peptide of proinsulin to the tumor site intra-arterially, intra-venously, subcutaneously, intra-peritoneally or by direct injection into the tumor bed, a very beneficial effect in terms of inhibiting blood supply to the tumor site and inducing tumor cell lysis is achieved simultaneously.

In clinical studies conducted by the inventor with PUFAs, the inhibition of blood supply was pronounced enough to cause cutting off blood supply to the tumor site with very little time lag. In other instances, an unmistaken strangling of blood supply to the tumor region was observed, but was relatively gradual. It was also noted that this occlusion of tumor feeding blood vessels is not by embolization, but is as a result of a chemical phenomenon which leads to constriction of the blood vessel walls as a result of interaction of the lithium salts of PUFAs+lymphographic agent+anti-angiogenic protein/peptide, 2-ME, H₂ blocker or C-peptide of proinsulin. Further, this phenomenon of occlusion of blood vessels was confined only to the tumor feeding blood vessels and is not seen when this combination of chemicals are given into the normal blood vessels. This came as a surprise finding to the inventor since this phenomena (selective occlusion of the tumor feeding blood vessels) was not observed when PUFA alone or PUFA (not a lithium or other salts of fatty acids)+lymphographic agent are given.

One aspect of the invention consists in the preparation of a combination/ composition of treatment of cancer in which one or more of LA, GLA, DGLA, AA, ALA, EPA, DHA and cis-parinaric acid are administered with conventional anti-cancer angents/drugs including anti-angiogenic protein/peptide, 2-ME, statins, H₂ blocker or C-peptide of proinsulin with or without an oily lymphographic agent or any other suitable agent for the delivery of these compounds; optionally, radiation may be included. The PUFAs may be provided in a daily dose of 0.5 mg to 100 gm together with appropriate doses of conventional anti-cancer drugs such as vincristine, doxorubicin, L-asparaginase, cis-platinum, busulfan, etc., in a daily/weekly/monthly dose of 1 mg to 50 gm depending on the requirement and the stage of the disease and as may be determined from time to time with or without the addition of anti-angiogenic protein/peptide such as angiostatin®/endostatin® in a dose of 1 mg to 100 mg/kg of body weight per day. The word anti-angiogenic substance as used herein includes one or more of the following substances such as: angiostatin®, endostatin®, platelet factor-4, TNP-470, thalidomide, interleukin-12, metalloprotease inhibitors (MMP), anti-adhesion molecules, 2-ME, statins, H₂ blockers and C-peptide of proinsulin (in their desired dose). The combination of PUFAs, conventional anti-cancer drugs, anti-angiogenic substances and the oily lymphographic agent may be administered by any one or different routes at the same time or at different times and intervals by selecting an appropriate route for each administration or in combination, eg. oral, parenteral including intra-arterial infusion, intravenous, subcutaneous, intra-peritoneal, topical, anal, vaginal routes as suppositories, or local injection directly into the tumor bed under the guidance of appropriate equipment such as but not limited to radiological guidance (X-rays), CT guidance or MRI guidance or by stereostaxic guidance. The daily dose(s) of these compounds may not exclude the administration of long acting preparations or depot preparation once or more times in a day, week, month or at some other appropriate time interval as determined from time to time depending on the necessity. The fatty acids (PUFAs) may be present in any physiologically acceptable form including but not limited to glycerides, esters, free acids, amides, phospholipids or salts. The conventional anti-cancer drugs may be administered by themselves or in conjugation with PUFAs (either alone or in combination such as GLA alone or GLA+AA, LA, DGLA, ALA, EPA or DHA). Similarly, 2-ME, statins, C-peptide, H₂ blocker, etc., may be given by themselves or in conjugation with PUFAs. For intra-arterial infusion or intravenous/subcutaneous injection/infusion or administration of LA, GLA, DGLA, AA, ALA, EPA, DHA and/or cis-parinaric acid may be given by themselves or in combination or dissolved or conjugated in/with 2-ME, statins, C-peptide of proinsulin, H₂ blockers etc., anti-angiogenic substances (such as angiostatin® and/or endostatin®) and in any other suitable solution that can be given parenterally but not limited to them. All these PUFAs, conventional anti-cancer drugs, anti-angiogenic substances including 2-ME, statins, C-peptide of proinsulin and H₂ blockers and lymphographic agent may each be given alone or in combination thereof or all together or separately at the same time or at different time intervals on the same day/week/month either by same route or different routes as the situation demands.

In order to observe or ascertain and record progress made in patients after administration of admixture according to this invention, images of the affected area eg., tumor region before and after treatment can be obtained by various known modalities such as computerized axial tomography (CT), magnetic resonance imaging (MRI), etc.

### EXAMPLES

1. Hard (wherein the PUFAs have been microencapsulated) or soft gelatin capsules (wherein the fatty acids are present in an oily form) made by accepted normal or forms or methods and are administered to persons suffering from cancer in conjunction with conventional anticancer drugs and/or anti-angiogenic substances or 2-ME, statins, C-peptide of proinsulin, and H₂ blockers including in the doses as stated supra.

2. Hard or soft gelatin capsules made by conventional methods, in which the fatty acids, the anti-cancer drugs and anti-angiogenic substances or 2-ME, statins, C-peptide of proinsulin or H₂ blockers are incorporated together in the same capsule and are administered to persons suffering from cancer.

3. As intra-tumoral preparation in appropriate doses (from 0.5 mg to 100 gm per day) of pure LA, GLA, DGLA, AA, ALA, EPA and DHA either individually or in combination thereof especially with anti-angiogenic substances or 2-ME, statins, C-peptide of proinsulin or H₂ blockers for the treatment of human brain gliomas or any other accessible tumor (eg. urinary bladdercancer, carcinoma of the esophagus, carcinoma of the lung, breast cancer etc.) by any route by using flexible fiber optic scopes such as bronchoscope, urethroscope, hysteroscope, etc. In the case of tumors of the head and neck the fatty acids are administered either by direct intra-tumoral route or by selective catheterization of the tumor feeding vessel(s) either by femoral, brachial or carotid routes or by subcutaneous route or intravenous route. The PUFAs and anti-angiogenic substances can be given to these patients daily, weekly or monthly or as and when necessary depending on the requirement and response of the patient to the treatment.

4. Administered as selective intra-arterial infusion or injection into the tumor feeding vessel by femoral, brachial or carotid routes or any other suitable route or in a combination thereof the PUFAs either alone or in combination with anti-cancer drugs/anti-angiogenic substances or 2-ME, statins, C-peptide of proinsulin or H₂ blockers with or without the oily lymphographic agent or any other suitable agent all in a mixture or in conjugated form(s) (like GLA+any conventional anticancer drug or drugs+anti-angiogenic substance, or 2-ME, statins, C-peptide of proinsulin or H₂ blockers, LA/GLA/DGLA/AA/ALA/EPA/ DHA/cis-parinaric acid all individually or in combination thereof+conventional anti-cancer drug(s)+anti-angiogenic substance(s)+lymphographic agent., LA/GLA/DGLA/AA/ALA/EPA/DHA/cis-parinaric acid in combination with or conjugated to anti-angiogenic substance(s) (including 2-ME, statins, C-peptide of proinsulin or H₂ blockers) or emulsified with or mixed with oily lymphographic agent., LA/GLA/DGLA/AA/ALA/EPA/DHA/cis-parinaric acid alone or in combination thereof in oily lymphographic agent as a mixture or emulsion or as a conjugate(s) and a variety of other combinations thereof). This preparation may be administered daily, weekly or monthly or at some other appropriate time interval.

5. Topical preparation of PUFAs either alone or in combination thereof with conventional anti-cancer drugs or anti-angiogenic substance(s) including 2-ME, statins, C-peptide of proinsulin, H₂ blockers in a suitable delivery vehicle in which daily doses (ranging from 0.5 μg to 100 mg) are applied to primary skin cancers including Kaposi's sarcoma locally and/or conventional anti-cancer drugs are given either orally or parenterally.

By the different embodiments of the invention method described supra, it becomes known that:

(i) when PUFAs are admnistered to patients intra-arterially or even otherwise as a combination with anti-angiogenic substance(s), 2-ME, statins, C-peptide of proinsulin or H₂ blockers there are less chances of albumin and other protiens binding to the fatty acids. Consequently, PUFAs and other chemicals thus administered using the invention are better available to be taken up by the tumor cells.

(ii) Owing to the efficient transportation of PUFAs and along with PUFAs the other chemicals such as the conventional anti-cancer drugs, anti-angiogenic substances, 2-ME, statins, C-peptide of proinsulin or H₂ blockers to the tumor site as described hereinbefore, there is increased intensity of the cytotoxic action of PUFAs and the administered anti-cancer agents (drugs or anti-angiogenic substance(s), 2-ME, statins, C-peptide of proinsulin, H₂ blockers or a combination thereof). Thus, using the invention, there is relatively better augmentation of free radical generation and lipid peroxidation in the tumor cells, thereby facilitating a greater degree of necrosis.

(iii) Inhibiting blood supply to the tumor region by the method of the invention prevents cell proliferation in the tumor region, thus enabling healthy tissue to grow back into place since, this method of treatment will not interfere with the normal blood vessles.

(iv) The inhibition otherwise caused by vitamin E and superoxide dismutase to free radical generation and lipid peroxidation produced by PUFAs, is reduced in the method of this invention because of the manner of transportation of PUFAs and consequently the anti-angiogenic substances, anti-cancer drugs, 2-ME, statins, C-peptide of proinsulin and/or H₂ blockers to the tumor site in the combination used intra-arterially through a proximate artery or intravenously or subcutaneously.

It is also within the purview of this invention, as stated supra to administer an admixture of PUFAs, anti-cancer drugs, and selected anti-angiogenic substance(s), 2-ME, statins, C-peptide of proinsulin and/or H₂ blockers at the same time, administering predetermined doses of PUFAs orally. All such variations are envisaged to be within the ambit of this invention.

Application to Mammals:

Even though the examples described supra relate to humans, it is envisaged that the method of inhibiting blood supply and using admixture of this invention including an anti-angiogenic substance, 2ME, statins, C-peptide of proinsulin or H₂ blockers are equally applicable to other mammals.

Equivalents

While this invention has been particularly shown and described with references to preferred embodiments thereof, it will be understood by those skilled in the art that various changes in form and details may be made therein without departing from the spirit and scope of the invention as defined by the appended claims. For example, anti-angiogenic substances referred to herein include not only Angiostatin® and Endostatin®, platelet factor-4, TNP-470, thalidomide, 2-ME, statins, C-peptide of proinsulin, H₂ blockers but other agents with anti-angiogenic and/or anti-cancer capabilities. Also sodium, potassium, ferrous, ferric, copper salts are considered equivalents of each other. Imaging techniques referred to herein are intended to include CAT, MRI, X-rays and other possible imaging methods. Those skilled in the art will recognize or be able to ascertain using no more than routine experimentation, many equivalents to the specific embodiments of the invention described specifically herein. Such equivalents are intended to be encompassed in the scope of the appended claims.

### REFERENCES

1. Battegay E J. Angiogenesis: mechanistic insights, neovascular diseases, and therapeutic prospects. J Mol Med 1995; 73: 333-346.

2. O'Reilly M S, Boehm T, Shing Y, Fukai N, Vasios G, Lane W S, Flynn E, Birkhead J R, Olsen B R, Folkman J. Endostatin: an endogenous inhibitor of angiogenesis and tumor growth. Cell 1997; 88: 277-285.

3. Sim B K, O'Reilly M S, Liang H, Fortier A H, He W, Madsen J W, Lapcevich R, Nacy C A. A recombinant human angiostatin protein inhibits experimental primary and metastatic cancer. Cancer Res 1997; 57: 1329-1334.

4. Bicknell R, Harris A L. Mechanisms and therapeutic implications of angiogenesis. Curr Opin Oncol 1996; 8: 60-65.

5. Gately S, Twardowski P, Stack M S, Patrick M, Boggio L, Cundiff D L, Schnaper H W, Madison L, Volpert O, Bouck N, Enghild J, Kwaan H C, Soff G A. Human prostate carcinoma cells express enzymatic activity that converts human plasminogen to the angiogenesis inhibitor, angiostatin. Cancer Res 1996; 56: 4887-4890.

6. O'Reilly M S, Holmgren L, Chen C, Folkman J. Angiostatin induces and sustains dormancy of human primary tumors in mice. Nature Med 1996; 2: 689-692.

7. O'Reilly M S. Angiostatin: an endogenous inhibitor of angiogenesis and of tumor growth. EXS 1997; 79: 273-294.

8. Wu Z, O'Reilly M S, Folkman J, Shing Y. Suppression of tumor growth with recombinant murine angiostatin. Biochem Biophys Res Commun 1997; 236: 651-654.

9. Gately S, Twardowski P, Stack M S, Cundiff D L, Grella D, Castellino F J, Enghild J, Kwaan H C, Lee F, Kramer R A, Volpert O, Bouck N, Soff G A. The mechanism of cancer-mediated conversion of plasminogen to the angiogenesis inhibitor angiostatin. Proc Natl Acad Sci USA 1997; 94: 10868-10872.

10.Lannutti B J, Gately S T, Quevedo M E, Soff G A, Paller A S. Human angiostatin inhibits murine hemangioendothelioma tumor growth in vivo. Cancer Res 1997; 57: 5277-5280.

11.Luo J, Lin J, Paranya G, Bischoff J. Angiostatin upregulates E-selectin in proliferating endothelial cells. Biochem Biophys Res Commun 1998; 245: 906-911.

12.Rivas M J, Arii S, Furutani M, Harada T, Mizumoto M, Nishiyama H, Fujita J, Imamura M. Expression of human macrophage metalloelastase gene in hepatocellular carcinoma: correlation with angiostatin generation and its clinical significance. Hepatology 1998; 28: 986-993.

13.Sang Q X. Complex role of matrix metalloproteinases in angiogenesis. Cell Res 1998; 8: 171-177.

14.Cornelius L A, Nehring L C, Harding E, Bolanowski M, Welgus H G, Kobayashi D K, Pierce R A, Shapiro S D. Matrix metalloproteinases generate angiostatin: effects on neovascularization. J Immunol 1998; 161: 6845-6852.

15.Lucas R, Holmgren L, Garcia I, Jimenez B, Mandriota S J, Borlat F, Sim B K, Wu Z, Grau G E, Shing Y, Soff G A, Bouck N. Pepper M S. Multiple forms of angiostatin induce apoptosis in endothelial cells. Blood 1998; 92: 4730-4741.

16.O'Mahony C A, Albo D, Tuszynski G P, Berger D H. Transforming growth factor-beta 1 inhibits generation of angiostatin by human pancreatic cancer cells. Surgery 1998; 124: 388-393.

17.Cao Y. Therapeutic potentials of angiostatin in the treatment of cancer. Haematologica 1999; 84: 643-650.

18.Ratel D, et al. Antibodies to endostatin in a multifocal glioblastoma patient. Lancet 2000; 356: 1656-1657.

19.Andre T, Chastre E, Kotelevets L, Vaillant J C, Louvet C, Balosso J, LeGall E, Prevot S, Gespach C. Tumoral angiogenesis: physiopathology, prognostic value and therapeutic perspectives. Rev Med Inteme 1998; 19: 904-913.

20.Mauceri H J, Hanna N N, Beckett M A, Gorski D H, Staba M J, Stellato K A, Bigelow K, Heimann R, Gately S, Dhanabal M, Soff G A, Sukhatme V P, Kufe D W, Weichselbaum R R. Combined effects of angiostatin and ionizing radiation in antitumour therapy. Nature 1998;

394: 287-291.

21.Gorski D H, Mauceri H J, Salloum R M, Gately S, Hellman S, Beckett M A, Sukhatme V P, Soff G A, Kufe D W, Weichselbaum R R. Potentiation of the antitumor effect of ionizing radiation by brief concomitant exposures to angiostatin. Cancer Res 1998; 58: 56865-5689.

22.Nguyen J T, Wu P, Clouse M E, Hlatky L, Terwilliger E F. Adeno-associated virus-mediated delivery of antiangiogenic factors as an antitumor strategy. Cancer Res 1998; 58: 5673-5677.

23.Chen Q R, et al. Liposomes complexed to plasmids encoding angiostatin and endostatin inhibit breast cancer in nude mice. Cancer Res 1999; 59: 3308-3312.

24.Lottering M L, Haag M, Seegers J C. Effects of 17 beta-estradiol metabolites on cell cycle events in MCF-7 cells. Cancer Res 1992; 52: 5926-5932.

25.Aizu-Yokota E, Susaki A, Sato Y. Natural estrogens induce modulation of microtubules in Chinese hamster V79 cells in culture. Cancer Res 1995; 55: 1863-1868.

26.Fotsis T, Zhang Y, Pepper M S, Adlercreutz H, Montesano R, Nawroth P P, Schweigerer L. The endogenous oestrogen metabolite 220 methoxyoestradiol inhibits angiogenesis and suppresses tumor growth. Nature 1994; 368: 237-239.

27.Mukhopadhyay T and Roth J A. Superinduction of wild-type p53 protein after 2-methoxyestradiol treatment of Ad5p53-transduced cells induces tumor cell apoptosis. Oncogene 1998; 17: 241-246.

28.Huang P, Feng L, Oldham E A, Keating M J, Plunkett W. Superoxide dismutase as a target for the selective killing of cancer cells. Nature 2000; 407: 390-395.

29.Das U N. Tumoricidal action of cis-unsaturated fatty acids and its relationship to free radicals and lipid peroxidation. Cancer Lett 1991; 56: 235-243.

30.Kumar G S and Das U N. Free radical dependent suppression of mouse myeloma cells by alpha-linolenic and eicosapentaenoic acids in vitro. Cancer Lett. 1995; 92: 27-38.

31.Cui X-L and Douglas J G. Arachdonic acid activates c-jun N-terminal kinase through NADPH oxidase in rabbit proximal tubular epithelial cells. Proc Natl Acad Sci USA 1997; 94: 3771-3776.

32.Tillotson J K, Darzynkiewicz Z, Chen L A, Ronai Z. Effects of linoleic acid on mammary tumor cell proliferation are associated with changes in p53 protein expression. Int J Oncology 1993; 3: 81-87.

33.Kastan M B, Canman C E, Leonard C J. P53, cell cycle control and apoptosis: implications for cancer. Cancer Metastasis Rev 1995; 14: 3-15.

34.Kumar K V, Das U N, Naidu K S. Effect of cis-unsaturated fatty acids on cell membrane bound enzymes in vitro. Nutrition Res 1992; 12: 549-560.

35.Mohan I K and Das U N. Effect of L-arginine-nitric oxide system on the metabolism of essential fatty acids in chemical-induced diabetes mellitus. Prostaglandins Leukotrienes Essen Fatty Acids. 2000; 62: 35-46.

36.Wahren J, et al. C-peptide revisited-new physiological effects and therapeutic implications. J Intern Med 1996; 240: 115-124.

37.Johansson B L, et al. C-peptide improves autonomic nerve function in IDDM patients. Diabetologia 1996; 39: 687-695.

38.Das U N. Essential fatty acids and osteoporosis. Nutrition 2000; 16: 386-390.

39.Das U N. Beneficial effect of eicosapentaenoic and docosahexaenoic acids in the management of systemic lupus erythematosus and its relationship to the cytokine net work. Prostaglandins Leukotrienes Essen Fatty Acids. 1994; 51: 207-213.

40.Rosenson R S, Tangney C C, Casey L C. Inhibition of proinflammatory cytokine production by pravastatin. Lancet 1999; 353: 983-984.

41.Gasparini G. The rationale and future potential of angiogenesis inhibitors in neoplasia. Drugs 1999; 58: 17-38.

42.Begin M E, Das U N, Ells G, Horrobin D F. Selective killing of tumor cells by polyunsaturated fatty acids. Prostaglandins Leukot Med 1985; 19: 177-186.

43.Begin M E, Das U N, Ells G. Cytotoxic effects of essential fatty acids (EFA) in mixed cultures of normal and malignant human cells. Prog Lipid Res 1986; 25: 573-577.

44.Begin M E, Ells G, Das UN, Horrobin D F. Differential killing of human carcinoma cells supplemented with n-3 and n-6 polyunsaturated fatty acids. J Natl Cancer Inst 1986; 77: 105

45.Das U N. Gamma-linolenic acid, arachidonic acid and eicosapentaenoic acid as potential anti-cancer drugs. Nutrition 1990; 6: 429-434.

46.Sangeetha P S and Das U N. Cytotoxic action of cis-unsaturated fatty acids on human cervical (HeLa) carcinoma cells in vitro. Prostaglandins Leukot Essen Fatty Acids 1995; 53: 287-299.

47.Das U N. Tumoricidal action of gamma-linolenic acid with particular reference to the therapy of human gliomas. Med Sci Res 1995; 23: 507-513.

48.Tsuchida T, Tsukamoto Y, Segawa K, Goto H, Hase S. Effects of cimetidine and omeprazole on angiogenesis in granulation tissue of acetic acid-induced gastric ulcers in rats. Digestion 1990; 47: 8-14.

49.Manjari V and Das U N. Effect of polyunsaturated fatty acids on dexamethasone-induced gastric mucosal damage. Prostaglandins Leukotrienes Essen Fatty Acids 2000; 62: 85-96.

50.Das U N. Hypothesis: Cis-unsaturated fatty acids as potential anti-peptic ulcer drugs. Prostaglandins Leukotrienes Essen Fatty Acids 1998; 58: 377-380.

51.Das U N, Kumar K V, Ramanjaneyulu E, Joshi N, Dixit V K. Essential fatty acids and their metabolites in duodenal ulcer. Med Sci Res 1994; 22: 210-213.

52.Venters H D, Tang Q, Liu Q, et al. A new meachanism of neurodegeneration: A proinflammatory cytokine inhibits receptor signaling by a survival peptide. Proc Natl Acad Sci USA 1999; 96: 9879-9884.

53.Lopez-Coviella I, Berse B, Krauss R, et al. Induction and maintenance of the neuronal cholinergic phenotype in the central nervous system by BMP-9. Science 2000; 289: 313-316.

1. A parenteral composition comprising 2-methoxyoestradiol (2-ME) 2-ME in any form including its structural analogues, and polyunsaturated fatty acids (PUFAs) in any form including a free acid, ester, phospholipid, glyceride, amides, and salts in amounts of from 0.1 mg to 100 gm 2-ME and from 0.5 mg to 100 gm PUFA(s), in a parenterally acceptable carrier.

2. The composition of claim 1 that is sterile and injectable.

3. The composition of claim 1 additionally comprising an osmolyte and a stabilizer and in a buffer at pH from 5 to 8.

4. A parenteral composition comprising 2-methoxyoestradiol (2-ME) in any form and polyunsaturated fatty acids (PUFAs) wherein the weight ratio of 2-ME to PUFA(s) in the composition ranges from about 1:10 to 10:1.

5. The composition of claim 1 wherein PUFAs may be one or more than one of the following: LA (linoleic acid), GLA (gamma-linolenic acid), DGLA (dihomo-gamma-linolenic acid), AA (arachidonic acid), ALA (alpha-linolenic acid), EPA (eicosapentaenoic acid), DHA (docosahexaenoic acid), cis-parinaric acid or conjugated linoleic acid.

6. A composition comprising of statin(s) and polyunsaturated fatty acids (PUFAs) in amounts of from about 0.1 mg to 100 gm of statin(s) and from 0.5 mg to 100 gm PUFA(s), in a parenterally acceptable form.

7. The composition of claim 6 that is sterile and injectable.

8. The composition of claim 6 additionally comprising an osmolyte and a stabilizer and in a buffer at pH from 5 to 8.

9. The composition of claim 6 wherein the weight ratio of statin(s) to PUFA(s) in the composition ranges from about 1:10 to 10:1.

10. The composition of claim 6 wherein statin includes one or more than one of the following: simvastatin, mevastatin, fluvastatin, lovastatin, pravastatin, or their derivatives or structural analogues and polyunsaturated fatty acids (PUFAs) may be one or more of the following: linoleic acid, gamma-linolenic acid, dihomo-gamma-linolenic acid, arachidonic acid, alpha-linolenic acid, eicosapentaenoic acid, docosahexaenoic acid, cis-parinaric acid and conjugated linoleic acid.

11. A method of administering the composition of claim 1 orally or parenterally, or an intra-arterial injection, which involves locating an artery which carries major blood supply to the tumor, said artery being one that is proximate to the tumor.

12. A method of administering the composition of claim 6, orally or parenterally or an intra-arterial injection, which involves locating an artery which carries major blood supply to the tumor, said artery being one that is proximal to the tumor.

13. A composition comprising of 2-methoxyoestradiol (2-ME) or its structural analogues or derivatives and polyunsaturated fatty acids (PUFAs) wherein an effective amount of 2-ME is complexed or conjugated with one or more of PUFA(s) such as: LA, GLA, DGLA, AA, ALA, EPA, DHA, cis-parinaric acid or conjugated linoleic acid.

14. A composition comprising of statins such as simvastatin, mevastatin, fluvastatin, lovastatin or pravastatin or their structural analogues or their derivatives and polyunsaturated fatty acids (PUFAs) wherein an effective amount of statin(s) is complexed or conjugated with one or more of PUFA(s) including: LA, GLA, DGLA, AA, ALA, EPA, DHA, cis-parinaric acid or conjugated linoleic acid.

15. A method of treating a cell proliferative disorder in a mammal comprising administering to the mammal an effective amount of the composition in claim 1 either orally, parenterally or by any other suitable route including intra-tumoral and/or intra-arterial injection or infusion.

16. A method of treating a cell proliferative disorder in a mammal comprising administering to the mammal an effective amount of the composition in claim 4 either orally, parenterally or by any other suitable route including intra-tumoral and/or intra-arterial injection or infusion.

17. A method of treating a cell proliferative disorder in a mammal comprising administering to the mammal an effective amount of the composition in claim 6 either orally, parenterally or by any other suitable route including intra-tumoral and/or intra-arterial injection of infusion.

18. A method of treating a cell proliferative disorder in a mammal comprising administering to the mammal an effective amount of the composition in claim 13 either orally, parenterally or by any other suitable route including intra-tumoral and/or intra-arterial injection or infusion.

19. A method as in claim 16 including an oily lymphographic agent as a carrier or a mixture or a conjugate.

20. A method as in claim 17 including an oily lymphographic agent as a carrier or a mixture or a conjugate.

21. A method as in claim 18 including an oily lymphographic agent as a carrier or a mixture or a conjugate.

22. The method of claim 15 wherein the cell proliferative condition may be selectively caused by angiogenesis and may include cancer, rheumatoid arthritis, systemic lupus erythematosus, psoriasis, scleroderma, corneal diseases, diabetic retinopathy associated with neovascularization, macular degeneration, ovulation, menstruation, keloids, uterine fibroids, diabetic nephropathy, osteoporosis, atherosclerosis, Alzheimer's disease and other dementias.

23. The method of claim 15 wherein the mammal is human.

24. The method of claim 15 additionally comprising administering to the mammal an effective amount of an anti-cancer agent/drug including radiation/radiotherapy.

25. The method of claim 16 wherein the cell proliferative disorder includes glioma (brain tumor).

26. A composition comprising H₂ blockers including cimetidine, ranitidine, famotidine and proton pump inhibitors of the type including omeprazole, lansoprazole and other similar derivatives and polyunsaturated fatty acids (PUFAs) may be one or more than one of the following: LA, GLA, DGLA, AA, ALA, EPA, DHA, cis-parinaric acid or conjugated linoleic acid.

27. The composition of claim 26 where in the H₂ blocker(s) or proton pump inhibitor(s) and polyunsaturated fatty acids (PUFAs) are present as a mixture or complex or conjugate.

28. The composition of claim 26 that can be given orally or parenterally in a suitable dose and form.

29. The composition of claim 26 wherein the ratio of H₂ blocker or proton pump inhibitor to polyunsaturated fatty acids (PUFAs) in the composition ranges from 1:10 to 10:1.

30. The composition of claim 26 comprising H₂ blockers or proton pump inhibitors and polyunsaturated fatty acids (PUFAs) in amounts ranging from about 10 mg to 100 gm H₂ blockers/proton pump inhibitors and from about 0.5 mg to 100 gm PUFAs, in a orally or parenterally acceptable form.

31. A method of treating a cell proliferative condition with or without angiogenesis in a mammal comprising administering to the mammal an effective amount of composition as recited in claim 26 either orally, parenterally or by any other suitable route including intra-tumoral and/or intra-arterial injection or infusion.

32. A method as in claim 31 adapted for treating a cancerous tumor including an oily lymphographic agent as a carrier or mixture or as a conjugate.

33. The method of claim 31 wherein the cell proliferative condition comprises one of cancer, rheumatoid arthritis, systemic lupus erythematosus, psoriasis, scleroderma, corneal diseases, diabetic retinopathy associated with neovascularization, macular degenration, ovulation, menstruation, keloids, uterine fibroids, diabetic nephropathy, osteoporosis, atherosclerosis, peptic ulcer disease.

34. The method of claim 31 wherein the mammal is human.

35. The method of claim 31 additionally comprising administering to the mammal an effective amount of an anti-cancer agent/drug including radiation/radiotherapy.

36. The method of claim 31 wherein the cell proliferative disorder can be glioma (brain tumor).

37. The method of claim 15 additionally comprising administering to the mammal an effective amount of a monoclonal antibody(ies) against tumor cell antigens tagged with or without cholera toxin or other toxins to which the composition of claim 1 is added, mixed or conjugated.

38. The method of claim 16 additionally comprising administering to the mammal an effective amount of a monoclonal antibody(ies) against tumor cell antigens tagged with or without cholera toxin or other toxins to which the composition of claim 6 is added, mixed or conjugated.

39. The method of claim 17 additionally comprising administering to the mammal an effective amount of a monoclonal antibody(ies) against tumor cell antigens tagged with or without cholera toxin or other toxins to which the composition of claim 14 is added, mixed or conjugated.

40. The method of claim 22 wherein the cell proliferative disorder can be glioma (brain tumor).

41. A composition comprising C-peptide of proinsulin, the carboxy-terminal tetra or penta peptides of the C-peptide and polyunsaturated fatty acids (PUFAs) may be any one or more than one of the following: LA, GLA, DGLA, AA, ALA, EPA, DHA, cis-parinaric acid or conjugated linoleic acid.

42. The composition of claim 41 wherein the C-peptide, the carboxy terminal tetra or penta peptides of the C-peptide and polyunsaturated fatty acids (PUFAs) are present as a mixture or complex or conjugate.

43. The composition of claim 41 that can be given orally or parenterally in a suitable dose and form, optionally including continuous intravenous infusion, if necessary, at an appropriate dose and form.

44. The composition of claim 41 wherein the ratio of C-peptide or its carboxy terminal tetra or penta peptides to PUFA(s) in the composition ranges from 1:10 to 10:1.

45. The composition of claim 41 comprising C-peptide or its carboxy terminal tetra or penta peptides and PUFAs in the amounts of from about 5 pg to 100 mg C-peptide or its carboxy terminal tetra or penta peptides and from about 0.5 mg to 100 gm PUFAs, in a orally or parenterally acceptable form.

46. A method of treating a hyperglycemia disorder in a mammal comprising administering to the mammal an effective amount of composition recited in claim 41.

47. The method in claim 46 wherein the hyperglycemic disorder is diabetes mellitus and its associated long-term complications including diabetic nephropathy, retinopathy, neuropathy, vasculopathy.

48. The method of claim 46 wherein the mammal is human.

49. The method of claim 46 additionally comprising administering to the mammal an effective amount of a hypoglycemic agent.
